(19) United States
(12) Patent Application Publication
(10) Pub. No.: US 2013/0108882 A1
(43) Pub. Date: May 2, 2013

(54) Plasticizers for Adhesive, Coating and Sealant Compositions Applied to Asphalt

(71) Applicant: Henry Company LLC, El Segundo, CA (US)

(72) Inventors: Jonathan T. Stuart, Lansdale, PA (US); William J. Werts, Glenolden, PA (US)

(73) Assignee: HENRY COMPANY LLC, El Segundo, CA (US)

(21) Appl. No.: 13/665,877

(22) Filed: Oct. 31, 2012

Related U.S. Application Data
(60) Provisional application No. 61/554,427, filed on Nov. 1, 2011.

(51) Int. Cl. C08K 5/101; C09D 175/04; C09D 171/00; B32B 11/04; C09J 171/00; C07C 69/02; C07C 69/34; C07C 69/76; C08K 5/11; C09J 175/04

(52) U.S. Cl. 428/489; 524/315; 524/306; 524/318; 560/231; 560/190; 560/103

(57) ABSTRACT

An asphalt-containing substrate having an adhesive, coating or sealant composition applied to the substrate surface and adhesive, coating or sealant compositions are described herein. The composition resists softening of the asphalt compound in the asphalt-containing substrate at elevated temperatures and includes at least one base material and at least one plasticizer. The plasticizer has is a low molecular weight compound and preferably includes an ester-containing reaction product of at least one glycerol and at least one carboxylic acid or a glycol and an aryl carboxylic acid. The composition base material is preferably a polymer or copolymer that includes polyurethanes, silylated polyurethanes, silylated polyethers, copolymers thereof, and blends thereof. Products containing the composition and a method of applying the composition to a substrate are also disclosed herein.

### CROSS REFERENCE TO RELATED APPLICATION

This application claims the benefit under 35 U.S.C. §119(e) to U.S. Provisional Patent Application No. 61/554,427, filed Nov. 1, 2011, the entire disclosure of which is incorporated herein by reference.

### BACKGROUND OF THE INVENTION

1. Field of the Invention

The invention relates to an adhesive, coating or sealant composition for application to asphalt surfaces, and also for use in construction products having asphalt substrates or layers.

2. Description of Related Art

Asphalt is used in a variety of applications including road construction and roofing. However, asphalt is very susceptible to degradation via weather and other chemicals that can attack and disintegrate the asphalt. Accordingly, liquid-applied adhesive products such as sealants and adhesively-attached solid barriers such as air and water barrier membranes can be applied to the asphalt to prevent degradation, provide adhesion, and/or to tie into existing and/or newly installed building products. The adhesive compositions applied to asphalt surfaces typically include a base material such as a polymer or copolymer and at least one plasticizer which serves to improve the softness and flexibility of the adhesive composition. The plasticizers embed themselves between the polymer chains thereby decreasing the glass transition temperature, reducing brittleness and stiffness, and improving processability.

Traditional plasticizers used in adhesive, coatings and sealant compositions are phthalate-based compounds. U.S. Pat. Reissue No. Re 41,586 teaches an adhesive material that includes a diisodecyl phthalate plasticizer. U.S. Pat. No. 5,387,623 describes an adhesive composition that includes dicyclohexyl phthalate and diphenyl phthalate plasticizers. Phthalate-based compounds, however, have been perceived as having a potentially negative effect on human health and the environment. Further, they soften and liquefy many asphaltic compounds.

Other adhesive compositions present in the art include high molecular weight polymers as plasticizers. For example, these compounds may include longer chain polyether-based polyalcohols (“polyether polyols”). These high molecular weight polymers can increase the viscosity of the adhesive compositions causing it to be difficult to use. High molecular weight polymers can also include many functional groups such as hydroxyls that can increase the adhesive tack. Adhesive compositions with excessive tack attract dirt and grow mold and mildew resulting in an aesthetically unpleasing product.

Another drawback of many adhesive plasticizers used with asphalt substrates is the potential migration of the plasticizer from the adhesive compositions into the asphaltic substrate over time resulting in softening of the asphalt. Plasticizers used in some adhesive compositions are compatible with asphalt because the compositions include an asphalt component. U.S. Patent Application Publication No. 2004/0172899 teaches an adhesive composition for adhering roof insulation and water-proofing materials which includes an asphalt component, a polymer and a plasticizer. U.S. Pat. No. 4,871,792 also discloses an adhesive composition that includes bitumen (the primary component of asphalt) and plasticizer.

U.S. Pat. No. 7,317,051 teaches a sealer and adhesive system that uses silylated polymers, a compatibilizer, and asphalt in the composition. With these and similar systems, the adhesive needs to be either compatibilized with the asphalt as in U.S. Pat. No. 7,317,051 or uses other components such as polyols that are already very compatible with asphalt as in U.S. Patent Application Publication No. 2004/0172899 or U.S. Pat. No. 4,871,792. When adhesive compositions containing asphalt-compatible plasticizers are applied to an asphalt surface, plasticizers or the asphalt-compatible components can migrate and soften the asphalt surface resulting in deformation of the surface.

Thus, there exists a need in the art for adhesive compositions that contain plasticizers or other components that do not tend to soften asphalt.

### BRIEF SUMMARY OF THE INVENTION

The present invention includes an asphalt-containing substrate having at least one surface and an adhesive, coating or sealant composition applied to the substrate surface. It further includes an adhesive, coating or sealant composition that resists softening of an asphalt-containing substrate to which it is applied up to a softening point of an asphalt compound in the asphalt-containing substrate.

The invention also includes an adhesive, coating or sealant composition that includes at least one base material and at least one plasticizer. The plasticizer has a molecular weight less than about 3,000 grams per mole and a structure according to formula (I),

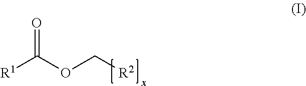

wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom, a saturated or unsaturated, branched or straight chain, functionalized or unfunctionalized alkyl group or ether-containing alkyl group having 1 to about 50 carbon atoms; or an ester-containing group. X is preferably from 1 to about 3. R¹ is preferably an alkyl group of about 1 to about 50 carbon atoms, but preferably has 1 to about 20 carbon atoms. R¹ may also be an alkanol group of preferably about 1 to about 20 carbon atoms. The R² alkyl group preferably has 1 to about 20 carbon atoms.

In a preferred embodiment, R¹ is a methyl group, and R² is an ester-containing group that is a reaction product of at least one alcohol having 1 to about 10 carbon atoms and at least one carboxylic acid having 1 to about 50 carbon atoms. In a more preferred embodiment, R² is an ester-containing group that is a reaction product of ethylene glycol and acetic acid.

In another preferred embodiment, R¹ is a fatty alcohol having about 8 to about 22 carbon atoms, and R² is an ester-containing group that is a reaction product of at least one alcohol having 1 to about 10 carbon atoms and at least one fatty acid having about 8 to about 22 carbon atoms. In a more preferred embodiment, R¹ is 9-heptadecen-7-ol and R² is an ester-containing group that is a reaction product of ethylene glycol and 12-hydroxy-9-octadecenoic acid.

In yet another preferred embodiment, R¹ is benzene, and R² is an ester-containing group that is a reaction product of at least one alcohol having 1 to about 10 carbon atoms and at least one aryl carboxylic acid having 7 to about 30 carbon atoms. R² is a more preferably an ester-containing group that is a reaction product of 2-methoxyethanol and benzoic acid.

In the most preferred embodiment, the plasticizer is oxydiethylene dibenzoate, castor oil, glycerol triacetate, and mixtures and combinations thereof.

The base material used in the adhesive, coating or sealant composition of the preferred embodiment may include a polymer or copolymer selected from polyurethanes, silylated polyurethanes, silylated polyethers, and copolymers thereof. The composition may also include at least one additive. The composition preferably includes about 10 to about 40 percent by weight of the base material, about 10 to about 20 percent by weight of the plasticizer, and about 40 to about 70 percent by weight of the at least one additive. In a further preferred embodiment, the adhesive composition resists softening of the asphalt-containing substrate up to no greater than about 140° F.

The present invention also includes products for use on asphalt-containing substrates that have the adhesive, coating or sealant compositions as described above. The products of the invention may include a liquid applied roofing product such as a water-proofing membrane, a white roof coating, a sealant, a mastic or a caulk. It may also be a applied to a building envelope comprising roofing, air and water barriers, a damp proofing product, or a roofing product

The invention further includes a method for applying the adhesive, coating or sealant compositions described above to an asphalt-containing substrate. The method includes applying the composition to an asphalt-containing substrate such as a roof to form at least one layer, wherein the composition resists softening of the asphalt-containing substrate up to a softening point of an asphalt compound in the asphalt-containing substrate, and in some preferred embodiments, resists softening up to no greater than about 140° F.

The invention further includes an asphalt-containing substrate having at least one surface and an adhesive, coating or sealant composition applied to the at least one substrate surface, wherein the adhesive composition resists softening of the asphalt-containing substrate up to a softening point of an asphalt compound in the asphalt-containing substrate, and the adhesive, coating or sealant composition comprises at least one base material and at least one plasticizer, wherein the at least one plasticizer has a molecular weight less than about 3,000 grams per mol and a structure according to formula (I),

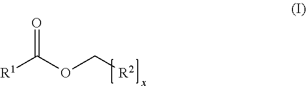

wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom; a saturated or unsaturated, branched or straight chain, functionalized or unfunctionalized alkyl group or ether-containing alkyl group having 1 to about 50 carbon atoms or an ester-containing group.

### DETAILED DESCRIPTION OF THE INVENTION

The present invention is directed to a sealed asphalt substrate that resists softening under elevated temperatures. It is further directed to an adhesive, coating or sealant composition useful for sealing asphalt which composition contains a base material and at least one low-molecular weight plasticizer. It will be understood by one skilled in the art based on this disclosure that the adhesive, coating or sealant compositions herein may be used on a variety of substrates, but find particular benefit on surfaces or products having asphalt therein, as the compositions resist softening of the asphalt when applied thereto.

The base material in the adhesive, coating or sealant compositions is preferably a polymer or copolymer and more preferably a polyurethane, a silylated polyurethane, a silylated polyether, copolymers of such polymers with other monomeric or polymeric species (either through random, block or graft copolymerization) or copolymers of such polymers with each other. In addition, blends or other combinations of these materials may be used.

The plasticizer in the compositions preferably has a molecular weight less than about 3,000 grams per mole and is preferably a reaction product of at least one glycerol and at least one carboxylic acid or a reaction product of at least one glycol and at least one aryl carboxylic acid. The plasticizer is more preferably oxydiethylene dibenzoate, castor oil, glycerol triacetate, and mixtures and combinations thereof. The invention is further directed to an adhesive product for an asphalt surface containing the plasticizer described above and a method for applying an adhesive composition containing the plasticizer described above.

With respect to the sealed asphalt substrates or adhesive, coating or sealant compositions used herein, asphalt includes any material primarily composed of natural or processed bitumen. Bitumen compounds are a class of viscous, solid, or semi-solid substances primarily containing high molecular weigh hydrocarbons. The asphalt may come in variety of forms including asphalt cement, emulsified asphalt, cutback asphalt, foamed asphalt, or mastic asphalt. An asphalt substrate is an asphalt-containing substance or medium to which another substance is applied. The asphalt substrates may have surfaces that may include, but are not limited to, surfaces of roads, bridges, parking lots, building surfaces such as roofs, waterproofing membranes, white roof coatings, or any other application where asphalt is used. Asphalt surfaces may also include the surfaces of asphalt-based products such as roofing shingles, asphalt membranes and asphaltic layers on laminated building products as well as building envelope systems having roofing, air and water barriers, damp proofing products, or other roofing products.

Concerning the adhesive compositions used herein, adhesive products include any products that have an active component(s) that can adhere or bond to surfaces. Adhesive compositions used in the adhesive products of the present invention include various types including both solvent-based and hot melt adhesives, or pressure-sensitive, heat-sensitive, moisture-reactive, drying, contact, or light-curing adhesives. The adhesive products, include, but are not limited to water-proofing membranes and other similar membranes, sealants, mastics, glues, caulks, and coatings. Coatings may be applied to any surface by any means and may cover all or part of the surface as desired. Sealants may be similarly applied in a manner such that the sealant composition fills, seals or otherwise covers cracks, crevices, holes, etc., in the substrate to which the composition is applied.

The adhesive, coating or sealant compositions herein include at least one plasticizer. The plasticizer is a low molecular weight compound that, when the composition is applied to an asphalt-containing substrate, the plasticizer resists softening the substrate at elevated temperatures. The plasticizer preferably has a low molecular weight (i.e., less about 3,000 grams per mole) to prevent a disadvantageous increase in the viscosity of the adhesive composition when mixed. The plasticizer more preferably has a molecular weight ranging from about 150 grams per mole to about 1,500 grams per mole. The plasticizer most preferably has a molecular weight ranging from about 200 grams per mole to about 950 grams per mole. Further, using the invention, the composition resists softening of an asphalt-containing substrate to which it is applied up to the softening point of an asphalt compound used as or in the asphalt-containing substrate. Such softening points may vary depending on the asphalt compound used. In certain embodiments, the composition resists softening of an asphalt-containing substrate to preferably no greater than about 140° F. (60° C.), but can, in some embodiments, resist softening at higher temperatures. The plasticizer also preferably does not include phthalate due to the perception of potential risks to human health and the environment associated with the phthalate compounds in addition to the tendency of phthalate-based plasticizers to migrate into the asphaltic substrate and soften the asphalt.

The plasticizer of the present invention is preferably represented by the general formula (I):

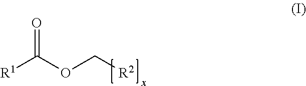

wherein in formula (I), x is an integer that ranges from 1 to about 10, preferably from 1 to about 3. R¹ may be a hydrogen atom or a saturated or unsaturated, functionalized or unfunctionalized, branched or straight chain alkyl group of 1 to about 50 carbon atoms, preferably 1 to about 20 carbon atoms. R¹ may also be a functionalized or unfunctionalized alkanol group having 1 to about 50 carbon atoms, preferably 1 to about 20 carbon atoms, wherein 18 carbon atoms provides castor oil. R¹ may further be a functionalized or unfunctionalized aryl group having about 6 to about 12 carbon atoms. Functional groups that may be used on R¹ include, but are not limited to aryl groups, aralkyl groups, fluoro, chloro, bromo, iodo, hydroxyl, carbonyl, aldehyde, haloformyl, carbonate ester, carboxylate, carboxyl, ether, ester, hydroperoxy, peroxy, caroxamide, amine, ketimine, aldimine, imide, azide, diimide, cyanate, isocyanate, nitrate, nitrile, nitrosooxy, nitro, nitroso, pydridyl, sulfonyl, sulfo, sulfinyl, sulfino, sulfhydryl, thiocyanate, disulfide, phosphino, phosphono, and phosphate groups. R¹ is preferably a fatty alcohol group having about 8 to about 24 carbon atoms, a saturated alkyl group having 1 to about 3 carbon atoms, or an aryl group having 6 to about 8 carbon atoms. R¹ is most preferably 9-heptadecen-7-ol, a methyl group, or benzene.

In formula (I), R² may be a hydrogen atom or a saturated or unsaturated, branched or straight chain, functionalized or unfunctionalized alkyl group having 1 to about 50 carbon atoms, preferably 1 to about 20 carbon atoms. R² may also be an ester-containing group and may further have additional ether linkages as well. Suitable functional groups for R² may include those listed above with respect to functional groups for R¹, but R² may be independently functionalized with different functional groups than those used on R¹. R² is preferably an ester-containing group which is a reaction product of at least one alcohol having 1 to about 10 carbon atoms, preferably 1 to about 3 carbon atoms, with one or more of the following reactant compounds: at least one carboxylic acid having 1 to about 30 carbon atoms, at least one fatty acid having about 8 to about 24 carbon atoms, or at least one aryl carboxylic acid having 7 to about 30 carbon atoms. R² is most preferably an ester-containing group which is the reaction product of ethylene glycol and acetic acid, ethylene glycol and 12-hydroxy-9-octadecenoic acid, or 2-methoxyethanol and benzoic acid.

Plasticizers of the present invention can also be formed so as to be an ester-containing reaction product of at least one glycerol and at least one carboxylic acid having about 2 to about 30 carbon atoms or at least one glycol and at least one aryl carboxylic acid having 7 to about 30 carbon atoms. More preferably, the plasticizer is formed as an ester-containing reaction product of glycerol and 12-hydroxy-9-octadecenoic acid, glycerol and acetic acid, or glycol and benzoic acid.

Thus, the most preferred plasticizers include oxydiethylene dibenzoate, castor oil, glycerol triacetate, and mixtures and combinations thereof. These preferred plasticizers have been shown to resist softening an asphalt surface at temperatures up to the standard softening temperature of the asphalt compound material in the asphalt surface, which may vary from compound to compound. In certain embodiments, it resists softening up to about no greater than about 140° F. (60° C.).

The composition also includes at least one base material as noted above. The base material may include any compound appropriate for use in an adhesive, coating or sealant composition, preferably at least one polymer. More preferably, the base material will include at least one of polyurethanes, silylated polyurethanes, silylated polyethers, and copolymers thereof. The copolymers may be random, alternating, block, or graft copolymers. Polyurethanes of the invention may be produced through a polymerization reaction where a monomer containing at least two isocyanate groups reacts with another monomer with at least two hydroxyl groups in the presence of a catalyst. A non-limiting example of a polyurethane of the invention is ECHELON™ MU 290 produced by the Dow Chemical Company of Midland, Mich. The silylated polyurethanes may be produced by the polymerization reaction of an amino- or sulfhydryl-functional silane with an isocyanate-terminated polymer or the reaction of an isocyanosilane with a polyol or a hydroxyl-terminated prepolymer. A non-limiting example of a silylated polyurethane of the invention is SPUR+™ produced by Momentive Performance Materials of Columbus, Ohio or Geniosil available from Wacker Silicones of München, Germany. The silylated polyethers can be produced by the reaction of a dimethoxysilane with an allyl-terminated polypropylene glycol. A non-limiting example of a silylated polyether of the invention is MS Polymer™ produced by Kaneka of Pasadena, Tex. These preferred base materials were selected for their resistance to weather, moisture, chemicals, corrosion, and cracking in addition to their compatibility with asphaltic compounds and general mechanical properties.

The adhesive, coating or sealant compositions herein may also include other additives. These additives may include one or more fillers including, but not limited to aluminium silicate, kaolin clay, aluminum oxide, limestone, barium sulfates, magnesium oxide, calcium carbonate, metal powder or flakes, potassium silicate, calcium silicate, silica, sodium silicates, ceramic beads, strontium sulfate/selestite, clays, talc or magnesium silicate, and dolomite. Other additives include light weight fillers like pumice, glass bubbles, polymeric bubbles and the like, adhesion promoters, desiccants, antioxidants, biocides, fungicides, catalysts, glass or cellulose fibers, pigments including, but not limited to carbon black and titanium dioxide, dyes, colorants, brighteners, thickeners, surfactants, tackifiers, UV absorbers, moisture absorbers, antistatic agents, mold release agents or other physical property modifiers. Preferably, the additives include calcium carbonate, dessicant, adhesion promoter, catalyst, and at least one pigment.

As discussed above, the adhesive, coating or sealant compositions of the present invention include, but are not limited to compositions including at least one base material, plasticizer, and additive. The base material is preferably at least one of polyurethanes, silylated polyurethanes and silylated polyethers, the plasticizer is more preferably oxydiethylene dibenzoate, castor oil, glycerol triacetate, and mixtures and combinations thereof, and the preferred additives include calcium carbonate, silica, and carbon black. The weight percent range of the base material in the adhesive is about 10 to about 60 percent by weight of the composition, preferably about 10 to about 40 percent. The weight percent range of the plasticizer is about 5 to about 30, preferably about 10 to about 20 percent by weight. The weight percent range of the additives is about 5 to about 85 percent by weight of the composition, preferably about 40 to about 70 percent by weight. The weight percent range of the calcium carbonate is about 0 to about 70 percent by weight of the composition, preferably about 40 to about 60 percent.

The products of the present invention may include, but are not limited to liquid products, particularly applied roofing products. The liquid applied roofing products preferably include waterproofing membranes, white roof coatings, sealants, mastics, or caulks. The adhesive compositions of the present invention may also be used to adhere membranes, such as water or air barrier membranes or roofing barrier tapes, and similar materials used in building envelope systems, and those which may be applied to asphalt-containing substrates, preferably by adhering to, coating and/or sealing the substrate surface, for example in a layered laminated building products structure having an asphaltic component or layer. The adhesive composition may be applied directly to the asphalt surface and/or to at least one surface of the membrane.

The present invention also includes a method for applying an adhesive, coating or sealant composition to an asphalt substrate, preferably via an upper or outer surface. The adhesive, coating or sealant composition can be applied to the asphalt surface using any suitable technique including rolling, spraying, or brushing to form at least one layer of any suitable thickness. The asphalt surface is preferably a building surface, and more preferably a roof. After the initial application of adhesive, coating or sealant composition, additional layers of such composition or other building or construction materials such as air or water barrier membranes, tapes, roofing shingles, etc. may be added or applied.

Various plasticizers were tested to determine whether they would soften asphalt surfaces under elevated temperatures. The non-limiting examples are described below:

### EXAMPLES

Multiple circular testing areas (“dams”) were constructed on an asphalt surface including 300 pen rubberized asphalt compound. About 20 milliliters of plasticizer were added to each dam. The asphalt surface was then placed in an oven and heated at a temperature of 120° F. for about 24 hours, and then up to 155° F. A rub test was then conducted to assess whether the plasticizers softened the asphalt surface. The asphalt area within each dam was then touched with a gloved finger. If the gloved finger showed a dark brown or black residue, the associated plasticizer failed the test.

The testing results are summarized in Table 1.

TABLE 1

| Manufacturer | Plasticizer | Rub Test |
|---|---|---|
| Vertellus ® Specialties of | Flexricin ® P8 | Fail |
| Indianapolis, Indiana | | |
| Vertellus ® | #1 Castor Oil | Pass |
| Vertellus ® | Polycin ® GR35 | Fail |
| Vertellus ® | Paricin ® 8 | Fail |
| Vertellus ® | Flexricin ® P1 | Fail |
| Vertellus ® | Flexricin ® P4 | Fail |
| Vertellus ® | Filtered Neutral Castor Oil | Pass |
| Vertellus ® | Citroflex ® A4 | Fail |
| Ferro of Cleveland, Ohio | Santicizer ® 278 | Fail |
| Ferro | Plaschek ® 775 | Fail |
| Ferro | Santicizer ® 261A | Fail |
| Lanxess of Germany | Mesamoll ® | Fail |
| Lanxess | Triacetin ® | Pass |
| Emerald Performance | Kflex ® DE | Pass |
| Materials of Cuyahoga Falls, | | |
| Ohio | | |
| Bayer Material Science of | Arcol ® PPG-3025 | Pass |
| Germany | | |

Mesamoll®, Santicizer® 278, and Santicizer® 261A are traditional, phthalate-based plasticizers and were tested for comparison purposes. Further, the Arcol® PPG-3025 is a high molecular weight plasticizer with 2 hydroxyl groups per chain. Thus, although the Arcol®PPG-3025 plasticizer passed the rub test, it was considered unsuitable for the present invention due to the goal of maintaining good viscosity and controlling tack of the adhesive composition.

Each plasticizer that passed the rub test was added to a basic sealant formula prepared having the following components: polyether polyol plasticizer (10-30 parts), calcium carbonate filler (30-60 parts), silylated polyether polymer (15-30 parts), additives including pigments, adhesion promoters, UV additives, and desiccants (15-50 parts) and retested to determine whether a sealant containing the exemplary plasticizer would resist softening of asphalt surfaces. For this test, multiple asphaltic compounds were used including Henry G100 S/S (asphalt base or protection sheet, rubber modified fiberglass reinforced). The sealant containing the plasticizer was applied to each asphalt surface using the same method described above and the asphalt surface was placed in an oven at a temperature of 120° F., and subsequently up to 155° for 7 days. A rub test was then conducted to assess whether the sealants containing the plasticizers softened the asphalt surface. All of the sealants containing a plasticizer according to the invention passed the rub tests.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An adhesive, coating, or sealant composition, the adhesive composition comprising at least one base material and at least one plasticizer, wherein the plasticizer has a molecular weight less than about 3,000 grams per mole and a structure according to formula (I), 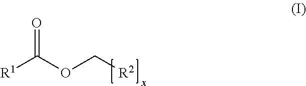
wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom; a saturated or unsaturated, branched or straight chain, functionalized or unfunctionalized alkyl group or ether-containing alkyl group having 1 to about 50 carbon atoms; or an ester-containing group.

2. The adhesive, coating or sealant composition according to claim 1, wherein the composition is capable of being applied at least one surface of an asphalt-containing substrate.

3. The adhesive, coating, or sealant composition according to claim 2, wherein the adhesive composition resists softening of the asphalt substrate up to a softening point of an asphalt compound in the asphalt substrate.

4. The adhesive, coating or sealant composition according to claim 3, wherein the adhesive composition resists softening of the asphalt substrate up to no greater than about 140° F.

5. The adhesive, coating or sealant composition according to claim 1, wherein R¹ is a methyl group and R² is an ester-containing group that is a reaction product of at least one alcohol having 1 to about 10 carbon atoms and at least one carboxylic acid having 1 to about 50 carbon atoms.

6. The adhesive, coating or sealant composition according to claim 5, wherein R² is an ester-containing group and is a reaction product of ethylene glycol and acetic acid.

7. The adhesive, coating or sealant composition according to claim 1, wherein R¹ is a fatty alcohol having about 8 to about 24 carbon atoms and R² is an ester-containing group that is a reaction product of at least one alcohol having 1 to about 10 carbon atoms and at least one fatty acid having about 8 to about 24 carbon atoms.

8. The adhesive, coating or sealant composition according to claim 7, wherein R¹ is 9-heptadecen-7-ol and R² is an ester-containing group that is a reaction product of ethylene glycol and 12-hydroxy-9-octadecenoic acid.

9. The adhesive, coating or sealant composition according to claim 1, wherein R² is an ester-containing group that is a reaction product of at least one alcohol having 1 to about 10 carbon atoms and at least one aryl carboxylic acid having 7 to about 30 carbon atoms.

10. The adhesive, coating or sealant composition according to claim 9, wherein R¹ is benzene and R² is an ester-containing group that is a reaction product of 2-methoxyethanol and benzoic acid.

11. The adhesive, coating or sealant composition according to claim 1, wherein the plasticizer is an ester-containing reaction product of glycerol and a carboxylic acid having about 2 to about 30 carbon atoms or an ester-containing reaction product of glycol and at least one aryl carboxylic acid having 7 to about 30 carbon atoms.

12. The adhesive, coating or sealant composition according to claim 11, wherein the plasticizer is a compound selected from the group consisting of oxydiethylene dibenzoate, castor oil, glycerol triacetate, and mixtures and combinations thereof.

13. The adhesive, coating or sealant composition according to claim 1, wherein the at least one base material comprises a polymer or copolymer.

14. The adhesive, coating or sealant composition according to claim 13, wherein the base material is selected from the group consisting of polyurethanes, silylated polyurethanes, silylated polyethers, copolymers thereof, and blends and combinations thereof.

15. The adhesive, coating or sealant composition according to claim 13, wherein the adhesive composition further comprises at least one additive.

16. The adhesive, coating or sealant composition according to claim 15, wherein the adhesive composition comprises about 10 to about 40 percent by weight of the base material, about 10 to about 20 percent by weight of the plasticizer, and about 40 to about 70 percent by weight of the at least one additive.

17. A product for use on an asphalt-containing substrate, comprising an adhesive, coating or sealant composition having at least one plasticizer, wherein the plasticizer has a molecular weight less than about 3,000 grams per mole and a structure according to formula (I), 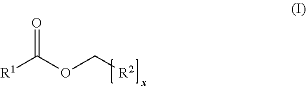
wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom; a saturated or unsaturated, branched or straight chain, functionalized or unfunctionalized alkyl group or ether-containing alkyl group having 1 to about 50 carbon atoms; or an ester-containing group.

18. The product according to claim 17, wherein the plasticizer comprises a compound selected from the group consisting of oxydiethylene dibenzoate, castor oil, glycerol triacetate, and mixtures and combinations thereof.

19. The product according to claim 17, wherein the adhesive, coating or sealant composition further comprises at least one base material and at least one additive.

20. The product according to claim 19, wherein the adhesive, coating or sealant composition comprises about 10 to about 40 percent by weight of the base material, about 10 to about 20 percent by weight of the plasticizer, and about 40 to about 70 percent by weight of the at least one additive.

21. The product according to claim 17, wherein the product is in a liquid form.

22. The product according to claim 21, wherein the product is applied to a building envelope comprising roofing, air and water barriers, a damp proofing product, or a roofing product.

23. The product according to claim 21, wherein the product is a water-proofing membrane, a white roof coating, a sealant, a mastic, or a caulk.

24. A method for applying an adhesive, coating or sealant composition to an asphalt-containing substrate, comprising applying an adhesive, coating or sealant composition to a surface of an asphalt-containing substrate to form at least one layer, wherein the adhesive, coating or sealant composition comprises at least one plasticizer with a molecular weight less than about 3,000 grams per mol and a structure according to formula (I), 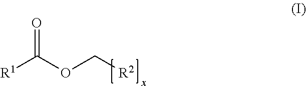
wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom; a saturated or unsaturated, branched or straight chain, functionalized or unfunctionalized alkyl group or ether-containing alkyl group having 1 to about 50 carbon atoms or an ester-containing group, wherein the adhesive, coating or sealant composition resists softening the asphalt-containing substrate up to a softening point of an asphalt composition in the asphalt-containing substrate.

25. The method for applying an adhesive, coating or sealant composition according to claim 24, wherein the adhesive, coating or sealant composition resists softening of the asphalt-containing substrate up to no greater than about 140° F.

26. The method for applying an adhesive, coating or sealant composition according to claim 24, wherein the plasticizer is selected from the group consisting of oxydiethylene dibenzoate, castor oil, glycerol triacetate, and mixtures and combinations thereof.

27. The method for applying an adhesive, coating or sealant composition according to claim 23, wherein the asphalt substrate is part of a building envelope comprising roofing, air and water barriers, a damp proofing product, or a roofing product.

28. An asphalt-containing substrate having at least one surface and an adhesive, coating or sealant composition applied to the at least one substrate surface, wherein the adhesive composition resists softening of the asphalt-containing substrate up to a softening point of an asphalt compound in the asphalt-containing substrate, and the adhesive, coating or sealant composition comprises at least one base material and at least one plasticizer, wherein the at least one plasticizer has a molecular weight less than about 3,000 grams per mol and a structure according to formula (I), 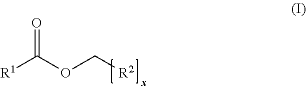
wherein R¹ is a hydrogen atom; a saturated or unsaturated, functionalized or unfunctionalized, branched or straight chain, alkyl group having 1 to about 50 carbon atoms, a functionalized or unfunctionalized alkanol group having 1 to about 50 carbon atoms or a functionalized or unfunctionalized aryl group having about 6 to about 12 carbon atoms; x is an integer from 1 to about 10; and R² is a hydrogen atom; a saturated or unsaturated, branched or straight chain, functionalized or unfunctionalized alkyl group or ether-containing alkyl group having 1 to about 50 carbon atoms or an ester-containing group.
